(19) United States
(12) Patent Application Publication
(10) Pub. No.: US 2024/0363206 A1
(43) Pub. Date: Oct. 31, 2024

(54) Imaging for Remote Lateral Flow Immunoassay Testing

(71) Applicant: Safe Health Systems, Inc., Scottsdale, AZ (US)

(72) Inventor: Ken Mayer, Los Angeles, CA (US)

(21) Appl. No.: 18/032,766

(22) PCT Filed: Oct. 19, 2021
(86) PCT No.: PCT/US2021/055588

§ 371 Date: Jun. 25, 2024

Related U.S. Application Data
(60) Provisional application No. 63/093,587, filed on Oct. 19, 2020.

(51) Int. Cl. G16H 10/40; G16H 15/00

(52) U.S. Cl. CPC G16H 10/40; G16H 15/00

(57) ABSTRACT

A computing system receives, from a user device, an image that depicts at least a readout area of a test strip from a lateral flow immunoassay (LFIA) test after an LFIA test has been performed by applying a biological sample of a user to the test strip. The system obtains context data that describes a context of the LFIA test for the user, analyzes the image and the context data to generate an interpretation of a result of the LFIA test, wherein the interpretation indicates a condition of the user measured by the LFIA test, and returns the interpretation of the result of the LFIA test to the user device.

### CROSS-REFERENCE TO RELATED APPLICATIONS

This application claims the benefit of U.S. Provisional Application Ser. No. 63/093,587, filed Oct. 19, 2020. The disclosure of the prior application is considered part of (and is incorporated by reference in) the disclosure of this application.

### BACKGROUND

Self-administered diagnostic test kits are available for consumer home-use, which facilitate collection of bodily material samples such as blood, urine, saliva and stool. The user can then apply the collected sample to a location on a lateral flow immunoassay (LFIA) test strip or cassette. After a waiting period for the sample to migrate to and react with all reagents present in one or more analytical zones of the test strip, the test result can be “read” by the user, according to instructions provided by the test supplier. The simplest method of “reading” the test result consists of visually determining the presence or absence of lines, markers or symbols on the surface of the strip or cassette, which is interpreted according to the instructions as a “Positive”, “Negative” or “Invalid” outcome for one or more specific test analytes. For other tests where a semi-quantitative result is sought, “reading” the test result requires the user to determine the “best match” of one or more test zones against a visual “scorecard” provided by the test supplier that displays a gradient of color hues, color densities or grayscale densities for each test zone. The test result for each analyte is indicated by a legend printed on the scorecard alongside each color hue or density.

### SUMMARY

This document describes systems, methods, devices, and other techniques for digitally reading LFIA diagnostic tests. A digital image of an LFIA test is captured via a mobile device, along with context data (e.g., metadata) related to the device and/or attributes of the digital image. The collected data can be analyzed using computer-based image processing routines to interpret a test result. In some implementations, interpretation of the test result includes detecting the presence or absence of visual indicators in an analytical or control zone of the test strip, such as a line or symbol whose purpose is used to indicate a binary value representing either a positive or negative test result. Moreover, the readout of the test strip can be obfuscated or otherwise made unreadable to the human eye. The user can then upload the image to the processing system, or submit the image to an application on the user device to interpret the test result. In some implementations, this approach can enable diagnostic tests to be conducted in a non-clinical location while providing for accurate analysis and further remote consultation by a licensed healthcare provider in any location. Advantageously, some examples of the disclosed techniques can enable remote mobile scanning and assessment of LFIA diagnostics than can be scaled to meet the testing demand of treating common illnesses and disease states and emergent pandemic health crises worldwide.

For some use cases, including but not limited to population epidemiological screening, medical clinical trial blinded testing, and military screening for mission-affecting conditions, systematically obfuscating or masking the test strip or cassette to disrupt the user's visual interpretation of the test result is beneficial to prevent behavioral reactions by the user or those in physical, social, familial or community proximity to the test subject.

Some implementations of the subject matter disclosed herein include methods performed a system of one or more computers. The method can include receiving, by a computing system and from a user device, an image that depicts at least a readout area of a test strip from a lateral flow immunoassay (LFIA) test after an LFIA test has been performed by applying a biological sample of a user to the test strip. The system can obtain context data that describes a context of the LFIA test for the user. The system can analyze the image and the context data to generate an interpretation of a result of the LFIA test, where the interpretation indicates a condition of the user measured by the LFIA test. The system returns an indication of the interpretation of the result of the LFIA test to the user device, which can then be presented to the user in an appropriate form (e.g., in a user interface of an application, in an email, a social media post, a web portal, or in a device notification).

These and other implementations can further include one or more of the following features. The interpretation can indicate whether the user, according to the LFIA test, is pregnant or has a particular disease. The interpretation indicates whether the user, according to the LFIA test, has a particular disease, and the particular disease can be one of COVID-19, strep infection, HIV, or colon cancer.

The user device can be a smartphone, a notebook computer, a desktop computer, a smartwatch, or a tablet computing device. The image can be captured with a camera of the smartphone or the tablet computing device.

The test strip can be disposed within a cassette when the LFIA test is performed, and at least one of the test strip or the cassette can include one or more fiducials. The image can include a depiction of the one or more fiducials and the readout area. The system can use the depiction of the one or more fiducials in the readout image to identify the readout area (and optionally multiple readout areas) of the test strip based on at least one of a pre-defined position, orientation, or scale of the one or more readout areas relative to the one or more fiducials. The test strip can include multiple readout areas, and each readout area can correspond to one of a control zone or an analytical zone of the test strip.

At least a part of the context data can be received by the computing system from the user device in connection with the test data.

The context data can include a device identifier that identifies a type of the user device or a camera of the user device that was used to capture the image. The computing system can further use the device identifier to access a device profile for the type of the user device or the camera identified by the device identifier, wherein the device profile includes imaging specifications about the type of the user device or the camera that bear on image properties of the image. Analyzing the readout image and the context data to generate the interpretation can include setting/adjusting an image processing routine for analyzing the readout image based on the imaging specifications.

The context data can include a test identifier that indicates a type of the LFIA test. The test identifier can be used to access a test profile corresponding to the type of LFIA test indicated by the test identifier. The test profile can describe information about how to interpret results for the indicated type of LFIA test. Analyzing the readout image and the context data to determine the interpreted result of the LFIA test can include implementing an image processing routine for analyzing the readout image based on the test profile.

The test profile can indicate at least one of a material or a construction property of the LFIA test strip, locations of one or more readout areas on the LFIA test strip, or mapping information that correlates visual attributes of the one or more readout areas with diagnostic information about the health condition of the user.

The context data can include environmental data that indicates at least one of a temperature or a humidity of the environment at a location where the LFIA test was conducted or the readout image was acquired. Analyzing the readout image and the context data to determine the interpreted result of the LFIA test can include adjusting an image processing routine for analyzing the readout image based on the temperature or the humidity of the environment.

The image can be received from the user device over the Internet.

The interpretation of the result of the LFIA test can indicate that the user tested positive for the health condition, tested negative for the health condition, or that the result of the LFIA test was inconclusive. The interpretation of the result of the LFIA test can indicate that the user tested positive for the health condition. The computing system can return, to the user device along with the interpretation, a recommended action for the user related to the health condition.

The recommended action can be customized according to at least one of a location of the user, a medical profile of the user, or a demographic category of the user.

The readout of the test strip can be obfuscated.

Some implementations of the subject matter disclosed herein include methods in which a readout image is acquired with a camera of a mobile device (e.g., a smartphone, notebook computer, or tablet computing device). The readout image shows a readout of a lateral flow immunoassay (LFIA) test. The mobile device identifies a context associated with the LFIA test, and transmits the readout image and the context data to a remote computing system. At a later time, the user device receives from the remote computing system, an interpreted result of the LFIA test, wherein the interpreted result was determined through analysis of the readout image and the context data.

Some implementations of the subject matter disclosed herein include methods comprising actions of acquiring one or more images that depict (1) an encoded patient identifier for a patient and (2) a medical test result for the patient; identifying, by a computing device, implicit context for the medical test result for the patient, wherein the implicit context is not entered by user input; associating, by the computing device, the one or more images and the implicit context; and transmitting, from the computing device to a remote service provider, the one or more images and data indicative of the implicit context for the medical test result for the patient.

Additional aspects of the disclosed subject matter include a system having one or more processors and one or more computer-readable media. The computer-readable media can be encoded with instructions that, when executed by the one or more processors (e.g., data processing apparatus), cause the one or more processors to perform any of the computer-based methods and processes disclosed herein.

### DESCRIPTION OF DRAWINGS

FIG. 1 depicts an operational environment of a digital diagnostic system for performing automated reading of LFIA test results.

FIG. 2 illustrates structures and operation of an example LFIA test strip.

FIGS. 3A-3C depict further structures and operation of an example LFIA test strip. FIGS. 4A-4B depict example photographs of a test readout from an LFIA test strip disposed in a cassette.

FIG. 5 is a flow diagram of an example process by which a user interacts with a mobile application to permit assessment of whether a rapid point of care test or virtual consultation is recommended.

FIG. 6 is a flow diagram of an example process by which a mobile application on a user's device guides a user to perform a diagnostic test with an LFIA test kit.

FIGS. 7A-7E depict flow diagrams of example processes for capturing and analyzing a readout of an LFIA test with computer vision techniques.

FIGS. 8A-8B depict flowcharts of an example process for submitting and processing medical test results along with coded patient identifiers (e.g., barcodes positioned adjacent to test results on a LFIA test strip or casing).

FIG. 9 depicts an example computing system that can be applied to implement aspects of the techniques described herein.

Like numbers and references between the drawings indicate like elements.

### DETAILED DESCRIPTION

This specification describes computer-based systems, methods, devices, and other techniques for home diagnostic testing and remote diagnostic test result interpretation. A digital image of a lateral flow immunoassay (LFIA) test can be captured with the camera of a mobile device, along with context or metadata related to the device and/or attributes of the digital image. The collected data can be analyzed using computer-based image processing routines to interpret a test result. In some implementations, interpretation of the test result includes detecting the presence or absence of visual indicators in analytical or control zones of the test strip, such as a line or symbol whose purpose is used to indicate a binary value representing either a positive or negative test result. Moreover, the readout of the test strip can be obfuscated or otherwise made unreadable to the human eye. The user can upload the image to the processing system, or submit the image to an application on the user device to interpret the test result.

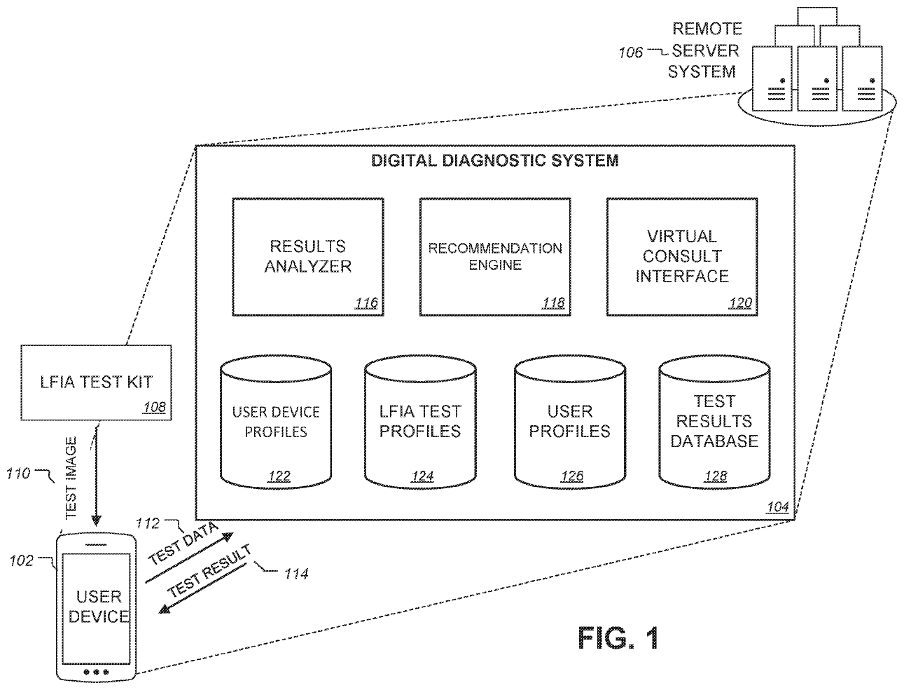

Referring to FIG. 1, an operational environment is depicted of a digital diagnostic system configured to perform automated reading of LFIA test results. The environment includes a user device 102, digital diagnostic system 104, remote server system 106, and LFIA test kit 108. The user device 102 and remote server system 106 can communicate over one or more networks, such as a local area network, wide area network, and/or the Internet. The digital diagnostic system 104 is typically implemented by the remote servers 106, but all or portions of the system 104 can alternatively be implemented locally at the user device 102. The LFIA test kit 108 is a diagnostic kit that uses LFIA to affect the appearance of a test strip based on the presence of one or more analytes in a biological sample/specimen from a user, which are typically biomarkers of a particular health condition. For example, certain antibodies in a bloodstream may indicate whether the user has or had a particular infectious disease.

Likewise, test strips that test for presence of human chorionic gonadotropin (hCG) can indicate whether the user is likely pregnant. The user can capture a photograph 110 (a readout image) of the test kit after performing a test, and upload the image along with context data as test data 112 to the digital diagnostic system 104. The digital diagnostic system 104 returns a test result 114 to the user device 102 based on analysis of the readout image and context data. In some implementations, the digital diagnostic system 104 implements a results analyzer 116 for performing image analysis routines to interpret a readout image, a recommendation engine 118 to generate a recommendation for the user based on the interpreted test result, a virtual consult interface 120 for facilitating scheduling a virtual consult between the user and a clinician. Various repositories 122-128 (e.g., databases) are also maintained at the digital diagnostic system 104 for storing user device profiles, LFIA test kit profiles, user/patient profiles (e.g., electronic health records), and test results including uploaded test data and test result interpretations. Additional detail on components of the digital diagnostic system are described below.

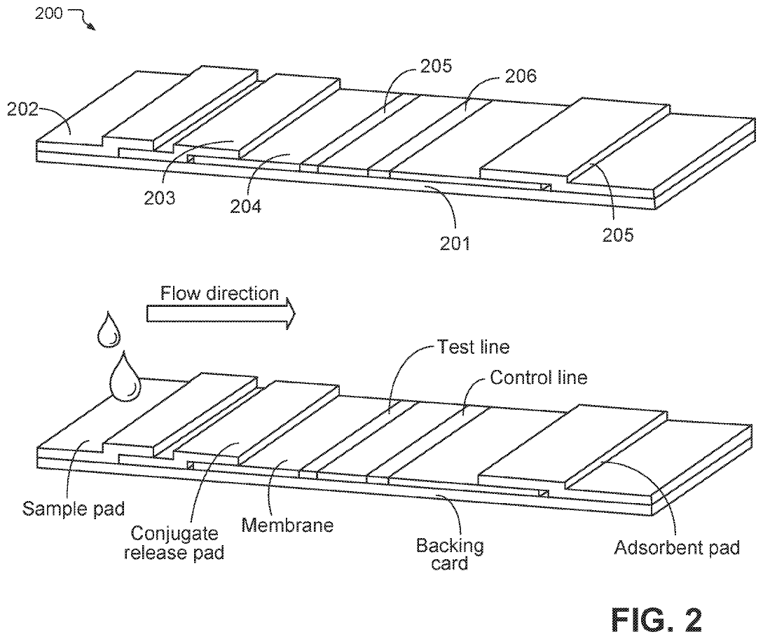

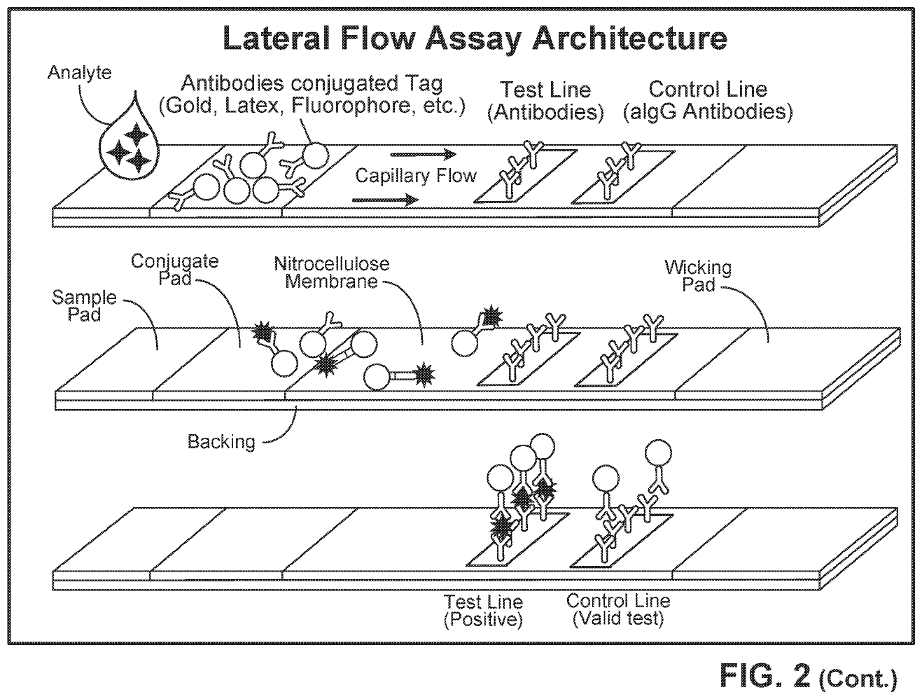

FIG. 2 depicts an example LFIA test strip 200 used for testing for the presence or absence of target analytes within a sample. The LFIA test strip 200 can have multiple components affixed to the top surface of a backing element 201 (e.g., a substrate) that provides structural support to the test strip. Non-limiting examples of the backing 201 include non-absorbent materials such as polymers, metals, or glasses. The components of the LFIA test strip 200 affixed to the backing 200 are arranged to transport a sample containing analytes through one or more reporting areas.

FIG. 2 depicts the LFIA test strip 200 with multiple components consisting of a sample pad 202, a conjugate pad 203, a capillary membrane 204, and an absorbent pad 205. The sample pad 202 can be an absorbent material where sample containing solutions are applied. The sample pad 202 abuts the conjugate pad 203, which stores one or more conjugating molecules. The conjugate pad 203 abuts the capillary membrane 204 responsible for the transportation of the sample containing solution across one or more reporting areas. The terminal absorbent pad 205 can abut the capillary membrane to facilitate sample wicking through the capillary membrane 204 and provide a reservoir in which to store sample containing solution after transport through the capillary membrane 204

In general, the capillary membrane 204 of the LFIA test strip 200 can have one or more reporting areas for reporting the presence of absence of a target analyte. For example, the target analyte can be an antibody, or antigen. For example, the reporting areas can show a visual signal in the presence or absence of the target analyte or control conjugate. This visual signal can include, but is not limited to, a color change, or the appearance of a shape, mark or symbol. For example, FIG. 2 depicts the LFIA test strip 200 as having two reporting areas in the shape of a line: a testing area 205 and a control area 206.

The capillary membrane 204 of the LFIA test strip 200 can have one or more separated channels for sample wicking. In some embodiments, the capillary membrane 204 can be divided into one or more disconnected sections aligned between the conjugate pad 203 and the absorbent pad 205. This can allow sample from the sample pad 202 to flow through the conjugate pad 203 and into the one or more capillary membrane 204 sections. Each capillary membrane 204 section can have one or more reporting areas corresponding to a different testing 205 or control 206 area.

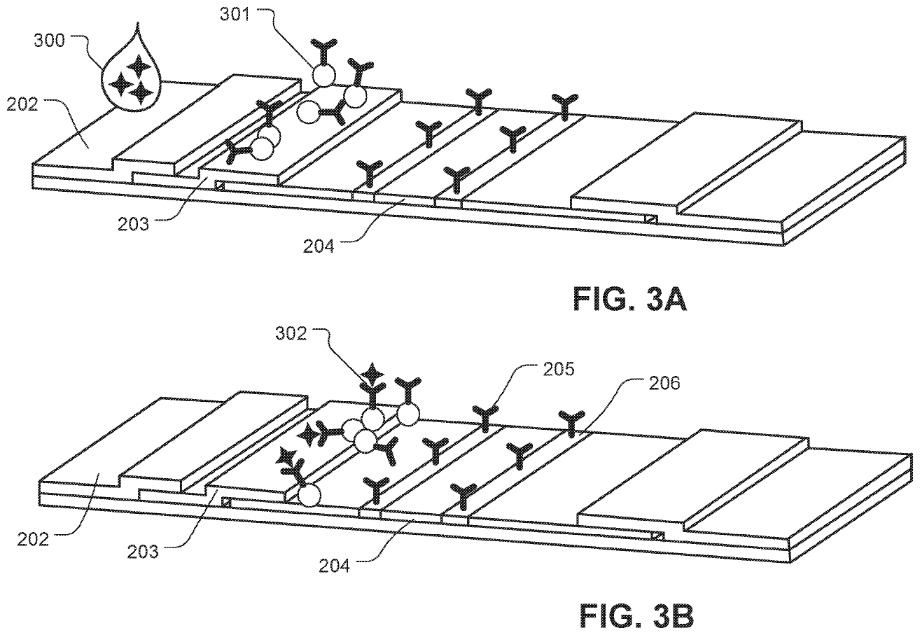

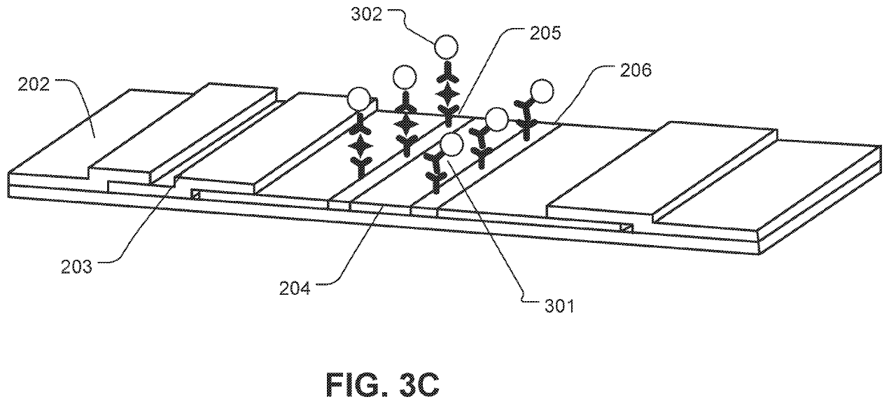

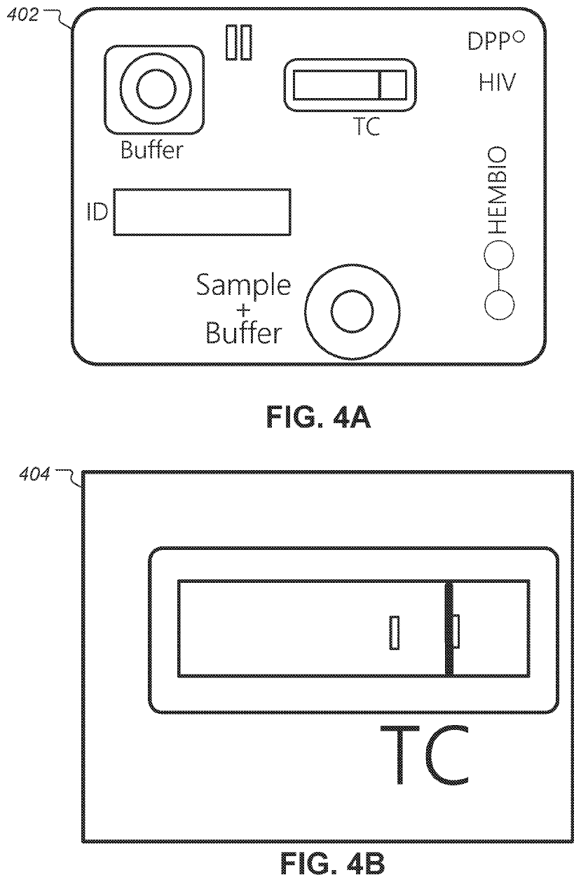

Referring now to the images of FIG. 3, an exemplary testing outline for the use of the LFIA test strip 200 is depicted. In FIG. 3A, a sample 300 containing a target analyte is shown being applied to the sample pad 202 of the LFIA test strip 200. The sample pad 202 can act as a sponge and can hold an excess of sample. Once the sample 300 has soaked the sample pad 202, the fluid can flow to the conjugate pad 203 in which one or more conjugates 301 are stored. The conjugate 301 can be an analyte binding agent (e.g., antibody) that has been specifically affixed with a reporter molecule. For example, the reporter molecule can be a molecule capable of providing a visual signal at high concentrations (e.g., colloidal gold). Further, the analyte binding agent of the conjugate 301 can be capable of forming a specific complex with the target analyte.

The conjugate pad 203 can further contain one or more reagents that enable a chemical reaction between the target analyte and the conjugate 301. As depicted in FIG. 3B, as the sample 300 containing the target analyte flows from the sample pad 202 to the conjugate pad 203, the conjugate 301 can specifically bind with the target analyte to form a complex 302. The conjugate pad 301 may contain more conjugate 301 than is necessary to bind the entire population of target analyte. Alternatively, the reaction between the conjugate 301 and the target analyte may not be efficient. For example, the entire population of target analyte may not bind to a conjugate 301 to form a complex 302 leaving excess target analyte and conjugate 301 in the sample solution. As the sample 300 flows through the conjugate pad 203, complex 302, excess target analyte, and unbound conjugate 301 can flow into the capillary membrane 204.

As described above, the capillary membrane can have one or more reporting areas. The reporting areas can contain additional analyte binding agent (e.g., antibody) that is immobilized within the one or more reporting areas of the capillary membrane 204. For example, FIG. 3B depicts the testing area 205 and control area 206 of FIG. 2. The immobilized analyte binding agents of the testing area 205 can be capable of specifically binding the target analyte. As the sample 300 containing complex 302, excess target analyte, and unbound conjugate 301 flows through the testing area 205, the target analyte of the complex 302 and excess target analyte can be specifically bound by the immobilized analyte binding agents. This can cause the complex 302 to become spatially concentrated. The increased spatial concentration of the reporter molecule in the complex 302 can cause a signal when the complex becomes immobilized in the testing area 205. The signal can be an indication of the presence of target analyte within the testing area.

In some embodiments, the positions of the reporting areas can be randomized. In some embodiments, the reporting areas can be an array of spots, lines, or other shapes. In some embodiments, the reporting areas can include more than one analyte binding agent. In some embodiments, the reporting areas can include more than one concentration of analyte binding agents. In some embodiments, the reporting areas can include permanent or temporary inks.

The sample 300 containing unbound conjugate 301 can continue to flow through the capillary membrane 204 to the control area 206 of FIG. 3B. The control area 206 can have a means of immobilizing the unbound conjugate 301 within the control area 206. For example, a population of target analyte can be immobilized within the control area with which the conjugate 301 can form a complex 302. For a further example, a target analyte competitor that is also capable of forming a complex 302 with the analyte binding agent of the conjugate 301 can be immobilized within the control area 206. The unbound conjugate 301 can become specifically bound in the control area 206. This can cause the unbound conjugate 301 to become spatially concentrated. The increased spatial concentration of reporter molecule in the control area 205 can cause a signal.

FIG. 3C shows how the result of the LFIA test strip 200 can be determined by the presence or absence of signal in the testing area 205, the control area 206, or both. FIG. 3C depicts bound complex 302 in the testing area 205 and bound conjugate in the control area 206. A signal in both the testing area 205 and the control area 206 may indicate the positive presence of the target analyte. A signal in the control area 206 and no signal in the testing area 205 can indicate a valid assay with a negative presence of the target analyte. An indeterminate signal in either the testing area 205 or control area 206 may indicate an inconclusive test (e.g., failed test).

A user can capture a digital image of a test strip, a cassette in which the test strip is disposed, or both, including any fiducial markers or identification data present on the strip or cassette for the purpose of facilitating alignment when interpreting the test result. The captured image has sufficient spatial resolution and color or grayscale fidelity to capture all readout necessary areas including analytical zones and control zones. Fiducial markers and test identification data can also be captured within the frame of the readout image to ensure that reliable and accurate test results can be obtained by appropriate analytical processing of the image.

The captured image can be processed using automated image analysis programs to determine the location and orientation of any fiducial markers or test identification data present on the test strip or cassette. If fiducial markers are identified in the readout image, the system can reference a profile of the test trip to determine a relative size, shape, and/or position of readout areas relative to the fiducial markers. In some implementations, image transformations such as rotation, scaling, sliding, cropping, skewing, or the like, can be performed to compensate for non-standard camera viewpoints used when acquiring the image. That is, if fiducial markers are present in the image, the relative position of the fiducial markers with respect to the known actual relative spatial positioning of other test strip or cassette features can be used to compensate for scale, orientation and tilt effects and/or distortions that may be present in the image of the test strip or cassette. In some implementations, the fiducial markers or other markers on at least one of the test strip can be encoded with information relevant to the interpretation of the test result (e.g., an encoding that uniquely identifies the type of test kit shown in the image). The system can use object detection or recognition routines to identify encoded markers in the image, and decode them accordingly.

Upon identifying relevant readout areas in the image, the system can analyze the readout areas (and, optionally, other areas of the image) to interpret the test and determine the presence or absence of analytical or control zones, lines, or symbols whose purpose is to indicate a binary value representing the test result. The readout image can be analyzed using computer-image processing, analysis, transformation and/or interpretation routines to determine the color hue, color density or grayscale density of any analytical or control zone, line or symbol whose purpose is to indicate a quantitative value.

The present system can also employ machine-learning techniques to facilitate interpretation of test results. For example, convolutional, recurrent, and/or feedforward neural networks can be trained to process at least a portion of a readout image along with certain types of context data to automatically generate an output that indicates a test result (e.g., positive, negative, or inconclusive). In some implementations, the neural networks or other machine-learning models are classifiers that process readout images as input, and output a classification corresponding to the detected test result.

The system can further process one or more forms of context data along with the readout image to interpret a test result. Context data is typically separate from the readout image itself, but can describe a number of relevant facets of the context in which the LFIA test was conducted or the context in which the readout image was obtained. For example, the context data can include data for device identification, camera settings, features and parameters, ambient, direct or flash lighting conditions, date and time, and environmental conditions such as temperature, relative humidity, geographical location, and geospatial orientation. In some implementations, the context data includes a visual marker that uniquely identifies the test subject. For example, the visual marker may be a 1D or 2D barcode (e.g., a QR-code) located so as to be visible in the image/photograph taken by the user. The identifier may be placed on the test strip, the cassette, or other devices within the test kit.

Once all information present has been decoded from fiducial or other visible markers, the system can determine a test result based on detected analytical or control zones, lines or symbols within an readout area whose purpose is to indicate a binary value, and quantitative values can be determined for any analytical or control zones, lines or symbols whose purpose is to indicate a quantitative value. In some implementations, one or more of the binary and quantitative values and their test strip or cassette image locations, and a-priori knowledge of the test strip or cassette characteristics can be used as an ensemble to determine the specific test result that was the target of the test procedure. For instance, the test strip or cassette can be produced with the analytical and control zones, lines and/or symbols obfuscated, e.g., through arrangement in a random order or location. A user lacking knowledge of the arrangement/ordering of the readout features, would be unable to properly interpret the test results. However, the arrangement/ordering of the readout features can be defined in a test profile stored by the system, and the system can reference the test profile to properly interpret the readout features according to the defined arrangement/ordering. In some implementations, all instances of a test of a particular type can have the same configuration (e.g., arrangement/ordering) of readout features. In other implementations, the configuration is dynamic and different instances of tests of the same type can have different readout configurations to further obfuscate. Test identifiers provided to the system can include a serial number or other unique identifier of the particular instance of the test, and the system can reference the identifier to access a corresponding test profile defined for that instance of the test.

The system can also use context to enable aggregate processing of groupings or sets of test results. Context data can include location data indicating a location where a test was performed or where the user acquired/uploaded a readout image. Location data can be used to determine recommendations or other supplemental information to return to a user in response to determining that the user tested positive for a particular condition (e.g., an infectious disease). For example, the system may identify and return indications of locations of facilities for monitoring, treatment, prescription delivery, or quarantine isolation nearby (e.g., within a threshold distance of) the test location.

In some implementations, the system can process images of non-obfuscated readouts to aid in interpretation despite the ability of many users to self-read the test. Automated interpretation using images can be beneficial when factors complicating manual reading are present such as test complexity, continuous or fine-grain gradient patterns, or user handicaps.

A mobile application on the user's device can guide the user through a specimen collection process, which could be urine, blood, saliva, epidermis, or epidermis swab for collecting material on the surface of the epidermis. LFIA paper test strips are used to screen for the presence of certain substances, including but not limited to germs, antibodies, hormones, bacteria, DNA, RNA, or viruses. The camera on a mobile phone or other user device can be used to photograph at least a portion of the LFIA test. In some implementations, by default, the photographed readout image of the LFIA test can be captured and stored at the maximum resolution supported by the camera and device.

The mobile application on the user's device can be configured to autofocus the camera's viewfinder on the test kit after a test has been conducted, and automatically capture the photograph when acceptable focus is achieved based on the end user's manual positioning of the device and lighting conditions as detected by a light sensor. Conditions such as the temperature and/or humidity of the environment can also be captured contemporaneously with the readout image. For example, environmental conditions can be identified with an API call to an online weather service.

In some implementations, the mobile application collects context data that facilitates automated interpretation of a readout image. A first example of context data is device data that indicates a type of the user device and/or camera that was used in capturing the readout image. A second example of context data is test kit data that identifies the test kit shown in a readout image. For instance, the test kit data can specify the make, model, SKU, serial number, expiration date, or a combination of these or other information about the test kit. A third example of context data is a timestamp that indicates a date, time, or both, for when the LFIA test was conducted or when the readout image was captured. A fourth example of context data is location data that indicates a location (e.g., geographic coordinates) where the LFIA test was conducted or where the readout image was captured. A fifth example of context data is environmental data that identifies information about the environment potentially bearing on interpretation of the test result. The environmental data can include, for instance, a temperature of the environment, a humidity of the environment, or both, at the time when and location where the test was conducted or the readout image was captured. Depending on the application, context data can be collected by the user device and sent to the digital diagnostics system for analysis, obtained by the digital diagnostics system based on other available context data, or both. For example, the user device may provide a timestamp and location identifier to the digital diagnostics system. The digital diagnostics system may then query a third-party weather service to obtain an indication of the temperature and/or humidity of the environment at the specified time and location. Lighting conditions can be detected by a light sensor of the user device, or inferred from time, location, or other context data. Some cameras automatically sample the natural lighting conditions of the environment to auto-adjust camera settings and determine whether to activate the flash. The readings from such sampling can be stored and transmitted as a form of lighting condition context to the digital diagnostics system.

Yet another example of context data that can be employed to facilitate interpretation of an image or generation of a proposed action or recommendation based on a test result is patient/user data. The patient/user data can include a patient record that indicates information about the patient/user whose biological sample/specimen was the subject of the test. For example, the patient record can indicate age, biological sex, prescribed medications (current or past), pre-existing conditions, allergies, family history, and social history, which can include data related to tobacco usage, drug usage, marital status, sexual orientation, condom use, etc. Patient/user data can be provided to the digital diagnostic system at the time of providing the readout image, or at an earlier time (e.g., during a pre-registration process). For example, patient/user data can be stored in a database of user profiles at the digital diagnostic system (e.g., user profile repository 126).

The digital diagnostic system maintains profiles for all supported user devices or cameras from which a readout image is obtained. User device profiles can be stored in a database, e.g., user device profiles repository 122. The profile for a given user device can include information such as the make, model number, SKU, schematics, default camera setting information, and OEM component identifiers, or a combination of these and other information about the user device and associated camera. For example, a user device profile may identify hardware or software specifications of the user device/camera, specifications for the camera lens, specifications for the light sensor of the device, camera resolution, and more.

The digital diagnostic system further maintains profiles for all supported test kits, and the profiles can be stored in a database (e.g., LFIA test profiles 124). The test kit profiles define information for each unique LFIA test that is necessary for the system to properly interpret readout images from a corresponding test. For instance, a test kit profile may include a unique identifier for the test kit that uniquely identifies the test. The system may compare an encoded identifier detected in a readout image to unique test kit identifiers from the various test kit profiles to determine a match to the appropriate test kit profile. A test kit profile may also define information about the location, size, shape, color, and/or orientation of readout areas and the corresponding readout features (e.g., analytical zones or control zones) in a specific area of the LFIA test strip. Further, information that maps visual attributes (e.g., shape, color/hue) of readout features, or combinations of readout features, to quantitative or qualitative interpretations of the test result for a given LFIA test can be defined in the profile for the corresponding test kit. A results analyzer of the digital diagnostics system can compare the detected visual attributes of readout features in the image to the mapped information to determine a quantitative or qualitative interpretation of the test result for a user (e.g., features that indicate a positive, negative, or inconclusive test, or features that indicate a quantitative assessment of a particular health condition). In some cases, the test kit profile defines compensation/adjustments that should be made to the test interpretation based on lighting conditions, humidity, temperature, or an amount of time elapsed between performance of the test and capturing of the readout image. The results analyze thus references a range of context data to maximize accuracy of the automated test interpretation. In some implementations, the test kit profile includes schematics or specifications describing properties of the construction or material of the test strip or cassette that bear on the system's analysis of a readout image. This can include specifications for the paper used to produce the test kit, schematics for the control used to produce the test kit, and more. A test kit profile can further identify the make, model, SKU, serial number, or combinations of these or other pertinent information about the test kit.

In some implementations, the system updates the models that map readout features to quantitative or qualitative interpretations of test results based on follow-up testing that either validates or rejects the initial interpretation determined by the digital diagnostic system. Follow-up/secondary testing may be done at the direction of the patient/user, a healthcare provider (e.g., physician), or at the recommendation of provider algorithms.

In some implementations, data received from a user device (including readout images and context data) and test result interpretations are stored in a test results database (e.g., database 128). The results database can be a relational database (e.g., SQL) or other suitable database from which data may be retrieved as needed. The results database may be centralized or distributed. In some implementations, information in the results database is stored in a distributed ledger (e.g., a blockchain).

In some implementations, if the system interprets a test as yielding an inconclusive result (e.g., not conclusively positive or negative), an assessment order ticket can be generated and routed to a selected healthcare provider. With the ticket, the healthcare provider can then access the test data and context data to manually review the readout from the test. Based on the manual review, the provider can enter an interpretation of the test result and the interpretation can be returned to the user. The fact that a provider provided manual review may or may not be revealed to the patient/user. In some implementations, a network of providers are registered with the digital diagnostics system. An assignment algorithm may be run to assign an assessment order ticket to a preferred provider. For example, the ticket can be dispatched to a particular provider in the network based on patient/user preferences, insurance requirements, proximity of the provider to the patient/user, type of test (e.g., pregnancy, colon cancer, COVID-19), quotas, amount of time since a provider was last assigned a ticket, or a combination of these and/or additional factors.

In some implementations, features from multiple readout areas on a test strip can be used to correct for lighting, comparative analysis and flow anomalies. For example, the system may analyze portions of the test strip depicted in a readout image located between analytical and control zones to correct for background anomalies such as incomplete latex (e.g., microparticle release) or low levels of sample amount or buffer solution. Additionally or alternatively, the system can take the ratio of color, hue, or darkness, or intensity of the control line and test line, to correct for lighting and quantitatively detect the amount of analyte being measured.

There are a wide variety of distinct variables between manufacturers and model of test strips, including but not limited to construction and composition of the components such as the backing card, adhesive strip, sample pad, and cover tape, that can affect the accurate reading and assessment of the test strip results. Construction techniques, the antibodies used, and buffers also play major role in the accurate reading and assessment of the test strip results. Governmental regulations often require test manufacturers to publish accurate product schematics for each test strip type that includes detailed information with regard to these variables. In some implementations, the digital diagnostic system can download these schematics and structure the data therefrom within a corresponding test kit profile. Information within the test kit profile can then be referenced to facilitate analysis of a readout image and interpretation of a test result.

The processes and system includes a method to collect and record the data provided in these schematics by either API interface, database upload function, or manually data entry based on the most efficient method available in each use case, including backing card, adhesive strip, sample pad, cover tape, and construction techniques, antibodies and buffers used in order to that can affect the accurate reading and assessment of the test strip results. This data is processed, along with all other meta data points collected at the time each test is taken, scanned and assessed, and the aggregate meta data that has been collected into the system from all previous tests residing in the system data lake, using machine learning and AI to calculate the variables for each particular test to ensure maximum accuracy of the assessment of the scan.

This multitude of LFIA test construction variables can be compounded by the multitude of variables apparent when sample collection is done by non-professionally trained users and the apparent variables associated with the reading of results from a photograph taken by a mobile device camera as opposed to a dedicated reader at the point of care. These variables can be parsed from the product schematics published by the LFIA test manufacture and included in the context data array. The aggregate data variables can be cross-referenced with the interplay between nanoparticle label type variables and all of the other variables that can effect each label type differently and processed using machine learning and AI to achieve the most accurate reading and assessment, including but not limited to temperature and environmental humidity of at the location where the same was collected and test strip scanned, which can have various effects on the colloidal stability in solution.

Referring to FIGS. 4A-4B, readout images 402, 404 are shown of an example LFIA test strip disposed in a cassette. The LFIA test can be conducted by a consumer at home, with the aid of instructions presented by a mobile device application. The user can then use the mobile device's integrated camera to photograph the LFIA test. In some implementations, user focuses on the entire test cassette (e.g., housing). The housing includes various markings which can serve as fiducials that indicate the relative position of the readout area in the image. FIG. 4A shows the cassette as a whole, while FIG. 4B is a view of the test kit zoomed in on the readout area in which analytical and control zones on the test strip are visible through an aperture of the cassette.

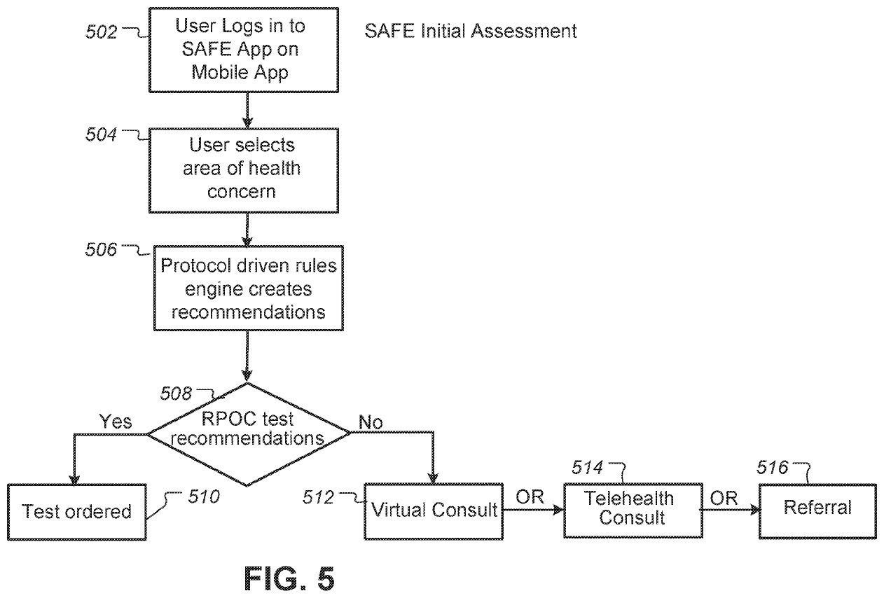

FIG. 5 is a flow diagram of an example process by which a user interacts with a mobile application to determine whether to order a rapid point of care diagnostic test or to perform a virtual consultation. A mobile application (referred to in the flowcharts as “SAFE app”) is installed on the user's mobile device. The user can launch and log into the application using appropriate credentials (502). Upon logging into the application, an interface is presented that lists various health areas (e.g., pregnancy, infectious diseases, cancers). The user can select a particular health area of concern (504), and a protocol-driven rules engine creates recommendations for the user based on information about the user's health status and health records (506). The mobile application further determines whether a rapid point of care test (RPOC), e.g., a LFIA test, is recommended for the user, and if so, the mobile application automatically orders a test kit (510). If an RPOC test is not recommended, the mobile application helps the user to schedule a virtual consultation (512), telehealth consultation (514), or generate a referral to a health care provider (516).

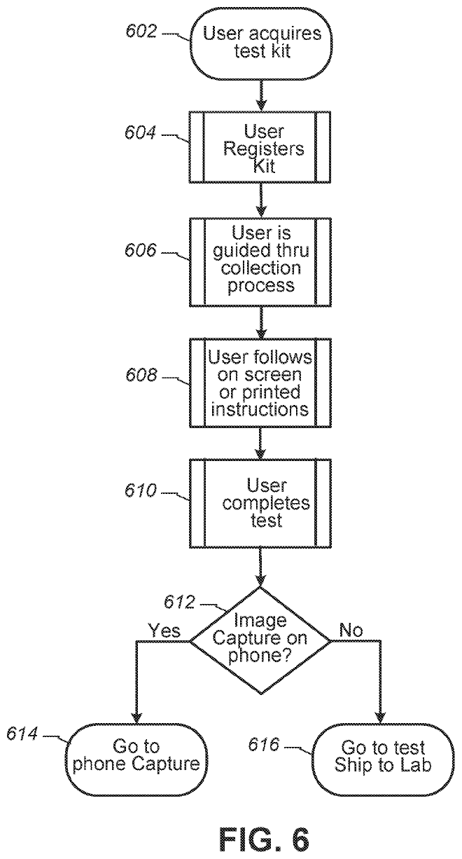

FIG. 6 is a flow diagram of an example process by which a mobile application on a user's device guides a user to perform a diagnostic test with an LFIA test kit. The user acquires a test kit (602), and registers the test kit through the mobile application on the user's device (604). For example, the user can enter information about the test kit such as its make, model, SKU, serial number, or the like, thereby providing the system with an association between the user's unique profile and the particular test kit employed. The mobile application can guide the user through a process for collecting a biological sample/specimen needed for the particular test, and how to deposit the sample/specimen on the test kit (606). The user follows the on-screen directions and/or printed instructions (608), and completes the test according to the instructions (610). If the user's device is equipped with a camera (612), the user launches the camera capability from the mobile application to capture an image of the test kit (614). If the user does not have the ability to capture an image of the test kit with her camera, the test kit can be shipped to a lab for analysis (616).

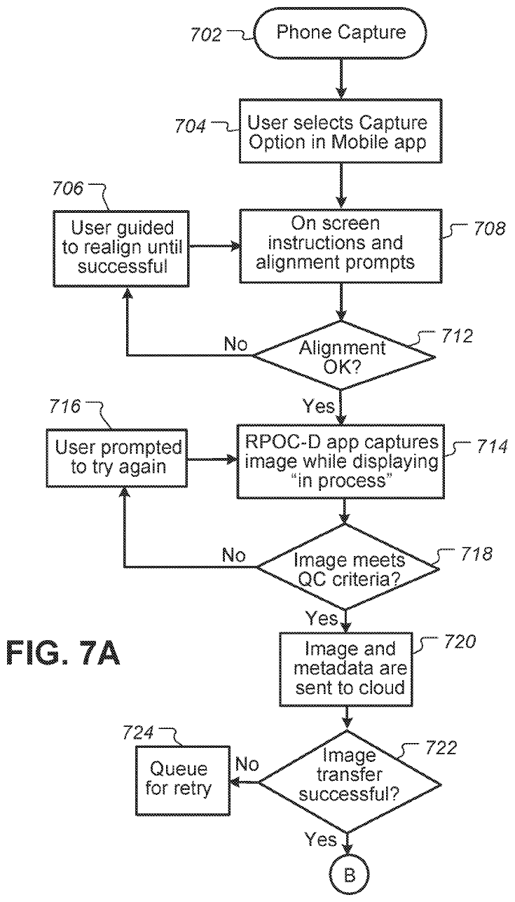

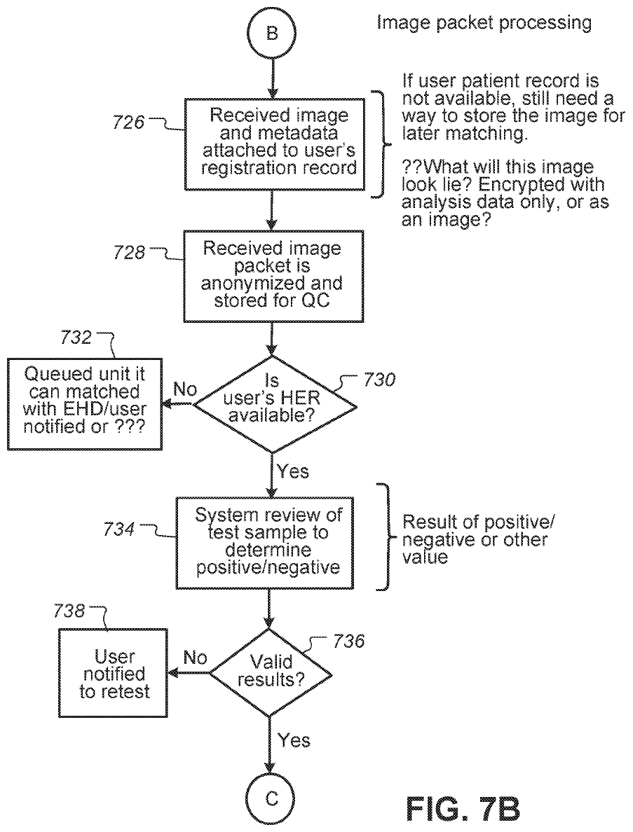

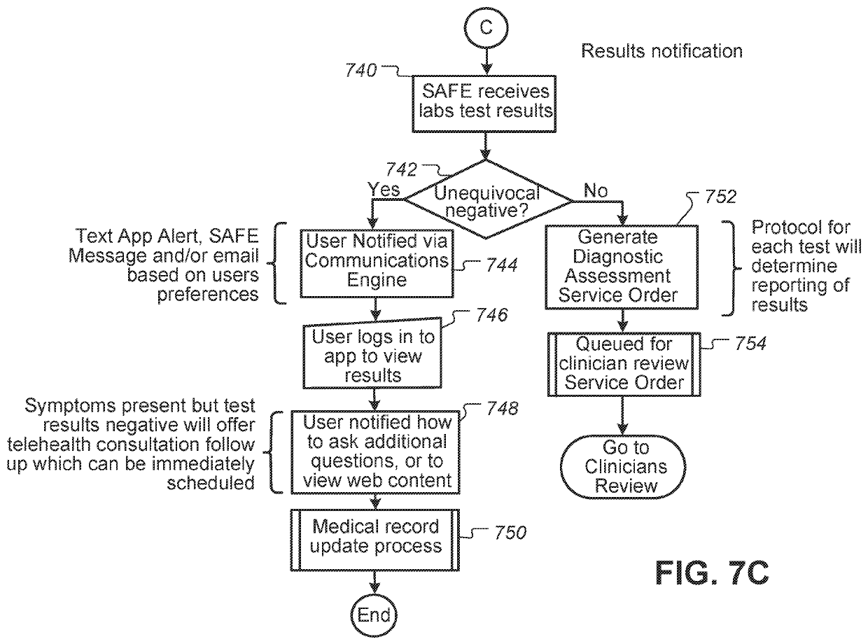

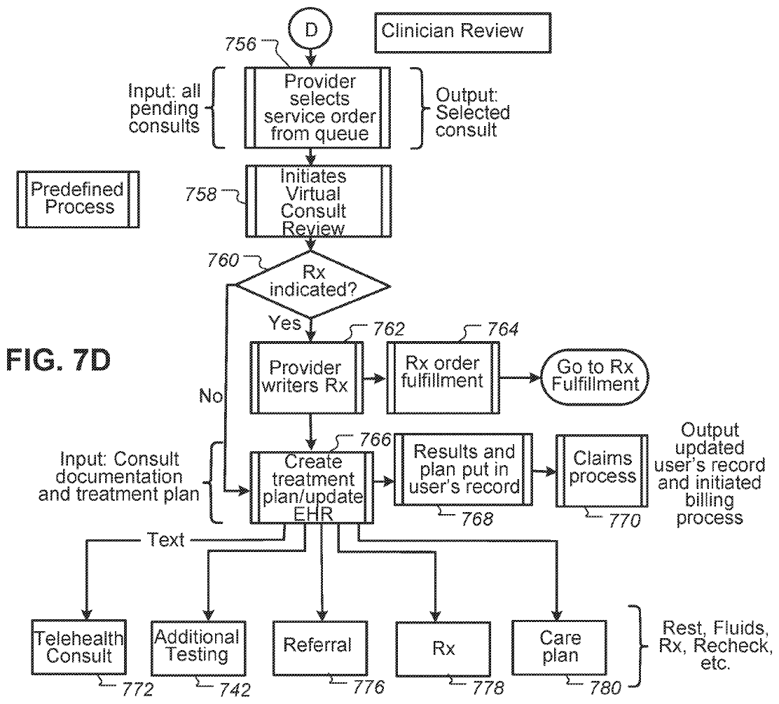

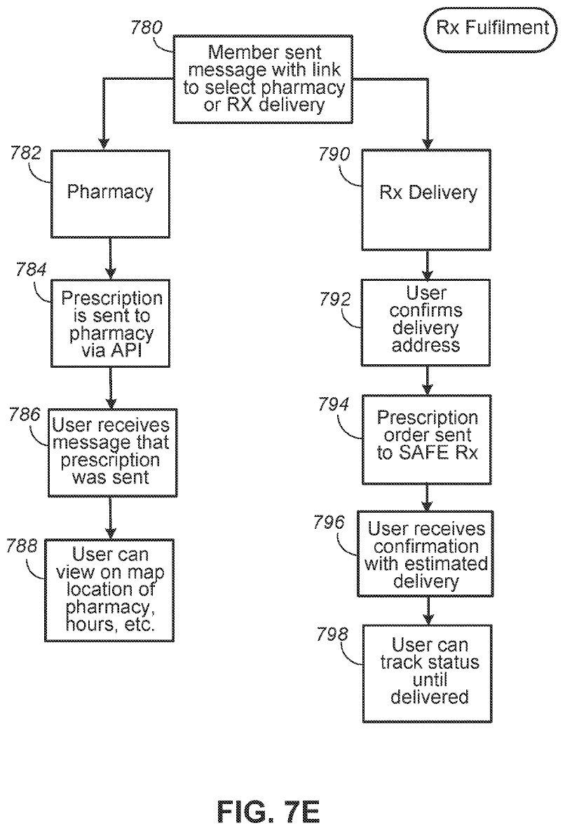

FIGS. 7A-7E depict flow diagrams of example processes for capturing and analyzing a readout of an LFIA test with computer vision techniques. If the user's device has a phone capture capability (702), the user selects the capture option in the mobile application (704). The application provides on screen instructions to facilitate alignment of the test kit in the viewfinder of the camera (708), and the user is guided to adjust alignment of the camera (708) until the test kit is sufficiently aligned and focused (712) and the readout image is captured of the test kit (714). The mobile application then assesses the captured image and determines whether it meets certain quality control criteria (718). If the image passes the quality control check, the device provides the image to a digital diagnostics system (720). If the image fails the quality control check, the user is prompted to re-take the photograph (716), and the process returns to 714. The digital diagnostics system can be implemented locally on the user's device, but typically is implemented by one or more remote servers as a cloud service. In the latter case, the readout image and associated context data relate to the image are uploaded from the device to the cloud service (720). If the image transfer is successful (722), the process proceeds, otherwise the upload can be retried (724).

The digital diagnostics system receives the readout image and associated context data (metadata) from the user device (726). To protect the privacy of the user, the received data can be blinded or anonymized, and stored in a test results database (728). The system can check if a user's electronic health record is available from a user profile database (730). If no EHR is available, the test results data can be queued for processing until the EHR is obtained (732). If the EHR is available, the system analyzes the readout image and context data to generate an interpretation of a result of the LFIA test (734). The system confirms whether the interpretation is valid (736). If the interpretation or test result is invalid, the user is notified and prompted to retest (738). If the interpretation is valid, the interpretation can be returned to a user along with a suggestion or recommended course of action corresponding to the test result. For instance, if the user tests positive for a particular condition, the user can be advised whether to self-quarantine, provided driving directions to a healthcare provider or pharmacy, or a virtual consultation can be scheduled. Alternatively, if a user had mailed the test kit for physical analysis rather than uploading an image of the test result, the service provider can retrieve the lab test results and an interpretation of the lab results is registered with the digital diagnostics system (740).

If the test result is an unequivocal negative (742), the user is notified via one or more communication channels such as text/SMS, app alert, or email (744). The user can log into the mobile application at his or her device to view the results (746). The user can also be notified how to ask additional questions, or to view web content. For example, if symptoms continue to present in the user despite the negative test result, the application may facilitate scheduling of a consultation follow-up (748). The user's electronic health record can be updated to reflect the negative test result (750). If the test result is not an unequivocal negative, the system can generate a diagnostic assessment service order (752). The interpretation of the test result is then queued for clinician review (754), and ultimately presented to a clinician (e.g., physician) for review. The clinician (provider) may access a portal where all queued service orders are listed in an interface. The clinician can select a particular service order (756). A virtual consult review can also be initiated (758). If a prescription is indicated (760), the provider can write a prescription (762), and the prescription fulfilled (764). If a prescription is not indicated, a treatment plan can be developed, and the patient's EHR updated (766). The results and plan can be stored in association with the patient's HER (user profile) (768), and a claims process initiated (770). The treatment plan can include a telehealth consultation (772), recommendation for additional testing (774), referral to a particular healthcare provider for an in-person appointment (776), writing of a prescription (778), and development of a care plan (780).

The prescription fulfillment process is illustrated in FIG. 7E. A member is sent a message (e.g., in the mobile application, by text or email) with a link to select a pharmacy for prescription fulfillment and pickup, or a prescription for delivery (780). If the user selects to have the prescription fulfilled at a local pharmacy (782), the digital diagnostics system sends to prescription to the user's selected pharmacy via API (784). The system can then message the user to confirm that the prescription order was sent to the pharmacy (786). The user can view information about the pharmacy, such hours of operation and location, online and through the mobile application (788). If the user selects to have the prescription delivered (790), the user can confirm his or her delivery address (792). The prescription order is sent to a pharmacy delivery service (794), the user receives confirmation with estimated delivery information (796), and the user can track order status until delivered (798).

Some aspects allow for rapid capture of clinical trial data based on ID or 2D barcodes and optical capture of test results such as lateral flow assays. The platform may thereby realize significant increases in efficiency of clinical trials by reducing the amount of time that research coordinators spend with patients, especially since the capture of safety testing would be rapid and prone to fewer data entry errors. A service provider administering the trial can then directly populate a database, including extraction of implicit metadata such as location of the interaction and the time/date of entry, and associate this metadata with the test result.

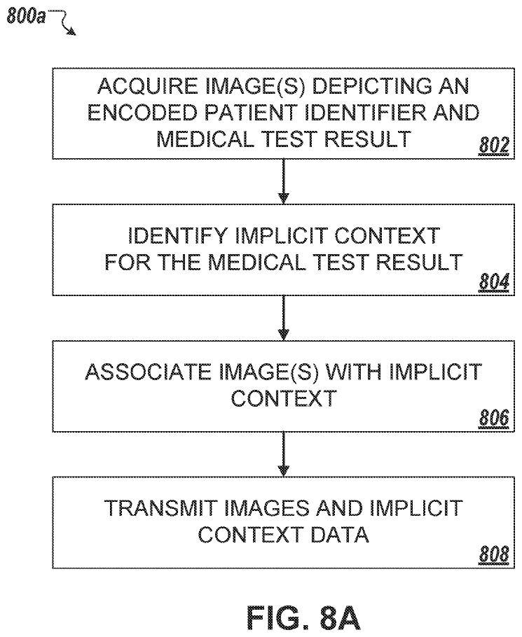

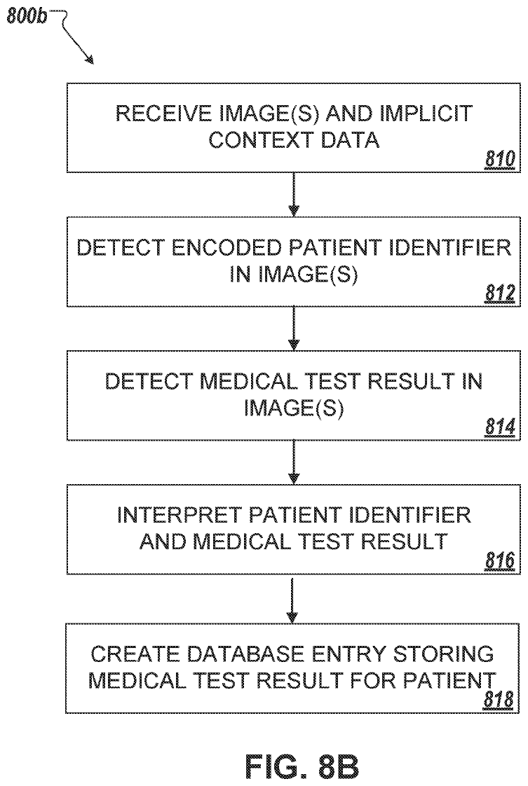

For example, FIGS. 8A-8B depict flowcharts of an example process 800a-b for submitting and processing medical test results along with barcoded patient identifiers. The system acquires one or more image captured by a camera (e.g., a camera of the tester's smartphone or other mobile device) (802). Either a single image, or multiple images collectively, show an encoded patient identifier and a medical test result for the patient. The encoded patient identifier can be a 1D or 2D barcode (e.g., a Quick Response (QR) code) that encodes information uniquely identifying the subject/patient of the test. The medical test result may be a picture of the result of a lateral flow immunoassay, for instance, or any other suitable test result. The tester's system (e.g., mobile device) identifies implicit context for the medical test result (804), such as the date/time the images and/or test was taken and the location. The system associates the acquired image(s) with the implicit context data, and provides them to a centralized test administrator (808). In some examples, the images and implicit context data are communicated over a network (e.g., the Internet) to a remote service provider's system.

Upon receipt of the image(s) and implicit context (810), the remote service provider's system analyzes the image and detects the encoded patient identifier (812) and the medical test result (814). Object detection and/or recognition techniques can perform operations 812 and 814 automatically, for instance. The system then interprets the patient identifier and medical test result (816). For example, the barcode image of the patient identifier can be decoded to recover a raw version of the patient identifier. Control lines of a lateral flow immunoassay test result can be analyzed to determine the test result, in some examples. The patient identifier and test result can then be stored in a centralized database for the clinical trial (818).

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, which is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

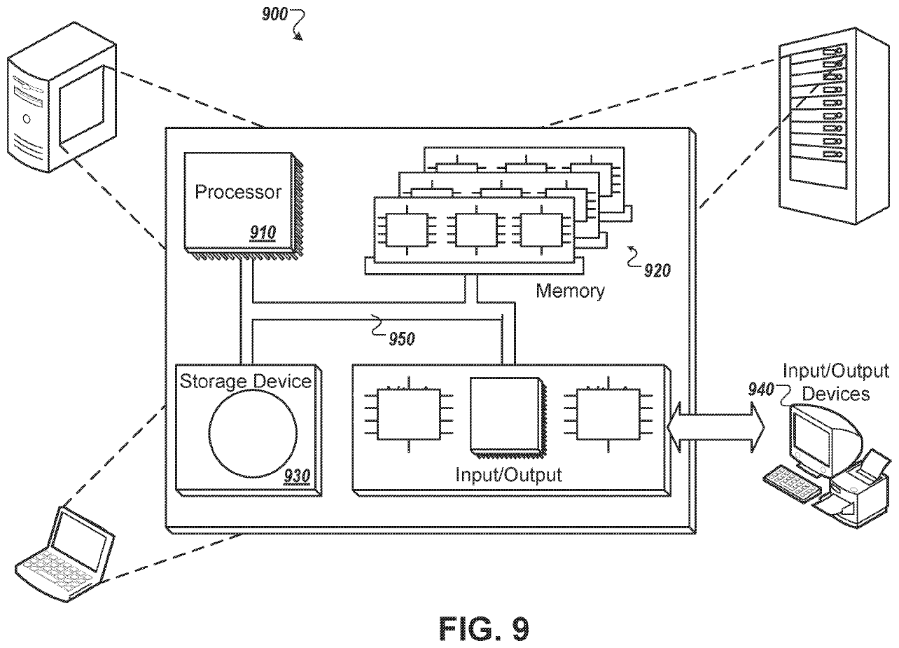

An example of one such type of computer is shown in FIG. 9, which shows a schematic diagram of a generic computer system 900. The system can be used for the operations described in association with any of the computer-implemented methods described previously, according to one implementation. The system 900 includes a processor 910, a memory 920, a storage device 930, and an input/output device 940. Each of the components 910, 920, 930, and 940 are interconnected using a system bus 950. The processor 910 is capable of processing instructions for execution within the system 900. In one implementation, the processor 910 is a single-threaded processor. In another implementation, the processor 910 is a multi-threaded processor. The processor 910 is capable of processing instructions stored in the memory 920 or on the storage device 930 to display graphical information for a user interface on the input/output device 940.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In one implementation, the storage device 930 is a computer-readable medium. In various different implementations, the storage device 930 may be a floppy disk device, a hard disk device, an optical disk device, or a tape device.

The input/output device 940 provides input/output operations for the system 900. In one implementation, the input/output device 940 includes a keyboard and/or pointing device. In another implementation, the input/output device 940 includes a display unit for displaying graphical user interfaces.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order consistent with logic and still achieve desirable results. As one example, the processes depicted in the accompanying figures do not necessarily require the particular order shown, or sequential order, to achieve desirable results. In some cases, multitasking and parallel processing may be advantageous.

Although various examples have been described in detail above, other modifications are possible. Accordingly, other implementations are within the scope of the following claims.

What is claimed is:

1. A computer-implemented method, comprising:
receiving, by a computing system and from a user device, an image that depicts at least a readout area of a test strip from a lateral flow immunoassay (LFIA) test after an LFIA test has been performed by applying a biological sample of a user to the test strip;
obtaining, by the computing system, context data that describes a context of the LFIA test for the user;
analyzing, by the computing system, the image and the context data to generate an interpretation of a result of the LFIA test, wherein the interpretation indicates a condition of the user measured by the LFIA test; and
returning, by the computing system, the interpretation of the result of the LFIA test to the user device.

2. The computer-implemented method of claim 1, wherein the interpretation indicates whether the user, according to the LFIA test, is pregnant or has a particular disease.

3. The computer-implemented method of claim 2, wherein the interpretation indicates whether the user, according to the LFIA test, has a particular disease, and the particular disease is one of COVID-19, strep infection, HIV, or colon cancer.

4. The computer-implemented method of clam 1, wherein the user device is a smartphone, a notebook computer, a desktop computer, a smartwatch, or a tablet computing device.

5. The computer-implemented method of claim 4, wherein the user device is a smartphone or a tablet computing device, and the image was captured with a camera of the smartphone or the tablet computing device.

6. The computer-implemented method of claim 1, wherein the test strip is disposed within a cassette when the LFIA test is performed, and at least one of the test strip or the cassette includes one or more fiducials;
wherein the image includes a depiction of the one or more fiducials and the readout area; and
wherein analyzing, by the computing system, the image and the context data to generate the interpretation of the result of the LFIA test comprises using the depiction of the one or more fiducials in the readout image to identify the readout area of the test strip based on at least one of a pre-defined position, orientation, or scale of the one or more readout areas relative to the one or more fiducials.

7. The computer-implemented method of claim 6, wherein the test strip includes multiple readout areas, each readout area corresponding to one of a control zone or an analytical zone of the test strip.

8. The computer-implemented method of claim 1, wherein at least a part of the context data is received by the computing system from the user device in connection with the test data.

9. The computer-implemented method of claim 1, wherein the context data includes a device identifier that identifies a type of the user device or a camera of the user device that was used to capture the image.

10. The computer-implemented method of claim 9, further comprising:
using the device identifier to access a device profile for the type of the user device or the camera identified by the device identifier, wherein the device profile includes imaging specifications about the type of the user device or the camera that bear on image properties of the image; and
wherein analyzing, by the computing system, the readout image and the context data to generate the interpretation comprises adjusting an image processing routine for analyzing the readout image based on the imaging specifications.

11. The computer-implemented method of claim 1, wherein the context data includes a test identifier that indicates a type of the LFIA test;
further comprising using the test identifier to access a test profile corresponding to the type of LFIA test indicated by the test identifier, wherein the test profile describes information about how to interpret results for the indicated type of LFIA test; and
wherein analyzing, by the computing system, the readout image and the context data to determine the interpreted result of the LFIA test comprises implementing an image processing routine for analyzing the readout image based on the test profile.

12. The computer-implemented method of claim 11, wherein the test profile indicates at least one of a material or a construction property of the LFIA test strip, locations of one or more readout areas on the LFIA test strip, or mapping information that correlates visual attributes of the one or more readout areas with diagnostic information about the health condition of the user.

13. The computer-implemented method of claim 1, wherein the context data includes environmental data that indicates at least one of a temperature or a humidity of the environment at a location where the LFIA test was conducted or the readout image was acquired; and
wherein analyzing, by the computing system, the readout image and the context data to determine the interpreted result of the LFIA test comprises adjusting an image processing routine for analyzing the readout image based on the temperature or the humidity of the environment.

14. The computer-implemented method of claim 1, wherein the image is received from the user device over the Internet.

15. The computer-implemented method of claim 1, wherein the interpretation of the result of the LFIA test indicates that the user tested positive for the health condition, tested negative for the health condition, or that the result of the LFIA test was inconclusive.

16. The computer-implemented method of claim 15, wherein the interpretation of the result of the LFIA test indicates that the user tested positive for the health condition;
further comprising returning, by the computing system and to the user device along with the interpretation, a recommended action for the user related to the health condition.

17. The computer-implemented method of claim 16, wherein the recommended action is customized according to at least one of a location of the user, a medical profile of the user, or a demographic category of the user.

18. The computer-implemented method of claim 1, wherein the readout of the test strip is obfuscated.

19. A system, comprising:
one or more processors; and
one or more computer-readable media having instructions stored thereon that, when executed by the one or more processors, cause the one or more processors to perform any of the methods of claims 1-18.

20. A computer-implemented method, comprising:
acquiring, with a camera of a mobile device, a readout image that shows a readout of a lateral flow immunoassay (LFIA) test;
identifying, by the mobile device, a context associated with the LFIA test;
transmitting, from the mobile device and to a remote computing system, the readout image and context data that describes the context of the LFIA test; and
receiving, from the mobile device and from the remote computing system, an interpreted result of the LFIA test, wherein the interpreted result was determined through analysis of the readout image and the context data.

21. One or more non-transitory computer-readable media having instructions stored thereon that, when executed by one or more processors, cause performance of the method of claim 20.

22. A mobile device, comprising:
one or more processors; and
one or more computer-readable media having instructions stored thereon that, when executed by the one or more processors, cause performance of the method of claim 20.

23. A method, comprising:
acquiring one or more images that depict (1) an encoded patient identifier for a patient and (2) a medical test result for the patient;
identifying, by a computing device, implicit context for the medical test result for the patient, wherein the implicit context is not entered by user input;
associating, by the computing device, the one or more images and the implicit context; and
transmitting, from the computing device to a remote service provider, the one or more images and data indicative of the implicit context for the medical test result for the patient.

24. The method of claim 23, wherein the encoded patient identifier comprises a one-dimensional barcode that encodes a unique identifier for the patient.

25. The method of claim 23, wherein the encoded patient identifier comprises a two-dimensional barcode that encodes a unique identifier for the patient.

26. The method of claim 25, wherein the two-dimensional barcode comprises a quick-response (QR) code.

27. The method of claim 23, wherein the computing device comprises a smartphone, a tablet computing device, a notebook computer, a desktop computer, or a wearable computing device.

28. The method of claim 23, wherein the medical test result comprises a result of a lateral flow immunoassay (LFIA) test.

29. The method of claim 23, wherein the encoded patient identifier and the medical test result are depicted in the same image.

30. The method of claim 23, wherein the encoded patient identifier and the medical test result are separately depicted in two or more images.

31. The method of claim 23, wherein the implicit context for the medical test result comprises a timestamp that indicates at least one of a date or time associated with the medical test result.

32. The method of claim 23, wherein the implicit context for the medical test result comprises an indication of a location where the medical test result was obtained.

33. One or more non-transitory computer-readable media having instructions stored thereon that, when executed by one or more processors, cause the one or more processors to perform any of the methods of claims 23-32.

34. A computing system, comprising:
one or more processors; and
one or more non-transitory computer-readable media having instructions stored thereon that, when executed by the one or more processors, cause the one or more processors to perform any of the methods of claims 23-32.

35. A computer-implemented method, comprising:
receiving, by a computing system, one or more images that depict (1) an encoded patient identifier for a patient and (2) a medical test result for the patient;
receiving, by the computing system and in conjunction with the one or more images, data indicative of an implicit context for the medical test result for the patient;
analyzing at least a first portion of the one or more images to interpret the encoded patient identifier;
analyzing at least a second portion of the one or more images to interpret the medical test result for the patient;
based on a result of interpreting the encoded patient identifier, accessing a patient record associated with the patient; and
storing an entry in the patient record for the patient that describes the interpreted medical test result for the patient and the implicit context for the medical test result.

36. The method of claim 35, wherein the encoded patient identifier comprises a one-dimensional barcode that encodes a unique identifier for the patient.

37. The method of claim 35, wherein the encoded patient identifier comprises a two-dimensional barcode that encodes a unique identifier for the patient.

38. The method of claim 37, wherein the two-dimensional barcode comprises a quick-response (QR) code.

39. One or more non-transitory computer-readable media having instructions stored thereon that, when executed by one or more processors, cause the one or more processors to perform any of the methods of claims 35-39.

40. A computing system, comprising:
one or more processors; and
one or more non-transitory computer-readable media having instructions stored thereon that, when executed by the one or more processors, cause the one or more processors to perform any of the methods of claims 35-39.
